(19) United States
(12) Patent Application Publication
(10) Pub. No.: US 2022/0400659 A1
(43) Pub. Date: Dec. 22, 2022

(54) METHOD FOR GENERATING STERILE ZEUGODACUS SCUTELLATA MALES BY USING ELECTRON BEAM IRRADIATION AND METHOD FOR CONTROLLING ZEUGODACUS SCUTELLATA BY USING SAME

(71) Applicant: ANDONG NATIONAL UNIVERSITY INDUSTRY- ACADEMIC COOPERATION FOUNDATION, Gyeongsangbuk-do (KR)

(72) Inventor: Yong Gyun KIM, Gyeongsangbuk-do (KR)

(21) Appl. No.: 17/780,215

(22) PCT Filed: Nov. 6, 2020
(86) PCT No.: PCT/KR2020/015478

§ 371 Date: May 26, 2022

(30) Foreign Application Priority Data
Nov. 26, 2019 (KR) 10-2019-0153762

(51) Int. Cl. A01K 67/033

(52) U.S. Cl. CPC A01K 67/0339; A01K 2227/706; A01K 2267/02

(57) ABSTRACT

The present invention relates to a method for generating sterile Zeugodacus scutellata males by emitting an electron beam at a dose of 150 Gy (inclusive) to 250 Gy (exclusive) to pupae of Zeugodacus scutellata and a method for controlling Zeugodacus scutellata by releasing the generated sterile males and normal males at a ratio of 9:1. In the present invention, electron beams are used instead of radioactive beams and suitable doses of electron beams are determined to generate sterile males of domestic native Zeugodacus scutellata. The generated sterile Zeugodacus scutellata males and normal males are released at a ratio of 9:1 to effectively control Zeugodacus scutellata through a sterile insect release technique (SIT).

### TECHNICAL FIELD

The present invention relates to a method of producing sterile males of Zeugodacus scutellata using electron beam irradiation and a method of controlling Zeugodacus scutellata by a sterile insect radiation technique using the produced sterile males of Zeugodacus scutellata.

### BACKGROUND ART

Due to climate change and increased international trade, new pests that did not exist in Korea have emerged (Lyu and Lee, 2017). Fruit flies, which are widely distributed mainly in Southeast Asia, are highly likely to invade and enter Korea due to such environmental changes (Kim and Kim, 2016), and 41 out of 60 import-prohibited pests designated by the Korean Animal and Plant Quarantine Agency are these fruit flies (Kim et al., 2017a).

Fruit flies that are considered to cause great damage to crops are mainly flies belonging to the large family Tephritidae, which includes more than 4,400 species (White and Elson-Harris, 1992; Norrbom et al.. 1999). There are 90 species of fruit flies that are native to Korea, and among these species, 4 species belong to the subfamily Dacinae, 4 species belong to the subfamily Phytalmiinae, 39 species belong to the subfamily Tephritinae, and 43 species belong to the subfamily Trypetinae (Han and Kwon. 2010; Han et al., 2014). Thereamong, fruit flies that cause economic damage to crops are two species: Zeugodacus scutellata and Z. depressa that belong to the subfamily Dacinae.

Zeugodacus scutellata grows in Trichosanthes kirilowii var. japonica Kitam in the wild, mainly in Jeju, but causes damage to pumpkins nationwide (Kim et al., 2017b). It was reported that Zeugodacus scutellata inflicts more damage to pumpkin flowers than pumpkin fruits, but damage to male flowers (53.8%) is greater than damage to female flowers (30.7%) (Kim et al., 2010). Zeugodacus scutellata adults are estimated to occur more than 4 times a year including the wintering generation (Al Baki et al., 2017; Kim et al., 2019). Zeugodacus scutellata is an insect pollinator, and since Zeugodacus scutellata is attracted to raspberry ketone (4-(4-hydroxyphenyl)-2-butanon) which is a plant-derived synomone, 4-(4-acetoxyphenyl)-2-butanon) (Cuelure, CL), a more stable compound, is used for outdoor monitoring of Zeugodacus scutellata (Kim et al., 2012, 2017b).

Since Zeugodacus scutellata larvae live inside pumpkin flowers, they are not exposed to sprayed chemicals and are difficult to control, and thus Zeugodacus scutellata adults in a developmental stage exposed outdoors are targets for control (Kim et al., 2017b). For example, based on lekking and pharmacophagy, which are shown by male fruit flies before mating (Hee and Tan, 2004), the male annihilation technique (MAT) using a mixture of Cuelure with an insecticide has been applied to control Zeugodacus scutellata (Kim et al., 2017b). In addition, many fruit fly females show anautogenous reproduction, which requires supply of nutrients through food to make eggs (Drew and Yuval, 2000), and based on this fact, a method of adding terpinyl acetate to the female annihilation technique (FAT) that uses a mixture of an insecticide and a protein attractant has been developed (Kim and Kwon, 2018).

An early monitoring and eradication program for foreign pests entering Korea is the basic direction of quarantine to protect domestic agricultural products.

When fruit flies subject to quarantine enter Korea, control technology using the male and female annihilation techniques is applied, but genetic control technology through sterile insect release can be applied to ultimately achieve an eradication effect (Kim et al., 2018a). The sterile insect release technique (SIT) is based on a control strategy that sterilizes males by irradiation and releases the sterilized males outdoors to mate with wild females to form unfertilized eggs (Knipling, 1955).

Several fruit fly species belonging to the Dacus subfamily, including Zeugodacus scutellata, are attracted to secondary metabolites secreted from Lilium flowers and show a specific group mating behavior called lekking, and thus the released sterile insects may sufficiently mate with wild insects. Accordingly, it was predicted that SIT would be very effective in controlling these fruit flies (Benelli et al., 2014, 2015).

SIT has been applied to control fruit flies in various regions. In fact, scientists from Hawaii and Australia applied SIT to completely eradicate Zeugodacus cucurbitae in Rota Island in the Northern Mariana Islands (Steiner et al., 1965), B. tryoni in Australia (Andreawartha et al., 1967), Bactrocera dorsalis in Micronesia (Steiner et al., 1970), and Ceratitis capitata in Hawaii (Harris et al., 1986). It was also reported that SIT was used to eradicate Zeugodacus cucurbitae in Japan (Koyama, 1996). In the case of Thailand, SIT was continuously applied to Bactrocera dorsalis, which had a great effect in reducing the size of the entire population (Aketarawong et al., 2011).

In the case of Bactrocera dorsalis, radiation was used to produce sterile males, and Bactrocera dorsalis pupa were left under hypoxic conditions 2 days before emergence and irradiated with a dose of 100 Gray (Gy) (Shelly et al., 2010). The sterile insects thus obtained are released either in the air or on the ground to the area where Bactrocera dorsalis occurs. For example, in the case of the SIT conducted in Hawaii, USA, 99,600 to 595,800 sterile males of Bactrocera dorsalis were released every week for about 8 months (Feb. 2 through Sep. 29, 2005), and a total of 11,556,000 sterile males were released (Vargas et al., 2010). However, when radiation is used to produce sterile insects, the harm caused by radiation emitting materials becomes a problem, and to this problem, a relatively safe sterile insect induction technology such as X-ray has been developed (Mastrangelo et al., 2010).

In addition, SIT has disadvantages in that mass breeding and subsequent irradiation reduces the mating ability of male adult insects and shortens their lifespan (Barry et al., 2003). To overcome the disadvantages of this genetic control technique, as another concept, the manipulation technique for the transformer gene, in which the characteristic post-transcriptional process occurs in the sexes during the sex determination period showing the secondary sexual characteristics, has been proposed as a strategy for the release of insects carrying a dominant lethal (RIDL) (Alphey, 2002; Fu et al., 2007). It was demonstrated that female lethal RIDL applied to Ceratitis capitata could have a successful eradication effect in a simple model experiment (Leftwich et al., 2014). However, this technique is expected to be difficult to apply outdoors because there is a concern about genetic disturbance in the ecosystem in that it releases transgenic insects produced using the transposon piggyBac.

### PRIOR ART DOCUMENTS

### Patent Documents

(Patent Document 1) Korean Patent No. 10-1976280

### Non-Patent Documents

(Non-Patent Document 1) Al Baki, Kim, H., Keum, E., Song, Y., Kim. Y., Kwon, K., Park, Y., 2017. Age grading and gene flow of overwintered Bactrocera scutellata populations. J. Asia Pac. Entomol. 20, 1402-1409.
(Non-Patent Document 2) Choi, D., Kwon, G., Kim, Y., 2018. Efficacy of wax-formulated lures on monitoring a quarantine insect pest, Zeugodacus caudata (Diptera: Tephritidae). Korean J. Appl. Entomol. 57, 185-190.
(Non-Patent Document 3) Han, H. Y., Kwon, Y. J., 2010. A list of North Korean Tephritoid species (Diptera: Tephritoidea) deposited in the Hungarian natural history museum. Korean J. Syst. Zool. 26, 251-260.
(Non-Patent Document 4) Han, H. Y., Choi, D. S., Rho, K. E., 2017. Taxonomy of Korean Bactrocera (Diptera: Tephritidae: Dacinae) with review of their biology. J. Asia Pac. Entomol. 20, 1321-1332.
(Non-Patent Document 5) Kwon, S., Choi, G. J., Kim, K. S., Kwon, H. J., 2014. Control of Botrytis cinerea and postharvest quality of cut roses by electron beam irradiation. Korean J. Hort. Sci. Technol. 32, 507-516.

### SUMMARY OF THE INVENTION

### Technical Problem

An object of the present invention is to provide a method of producing effective sterile males of Zeugodacus scutellata by analyzing the development of oocytes and sperm depending on each adult development period through analysis of the reproductive development process of Zeugodacus scutellata, which is native to Korea, in order to apply the sterile insect release technique (SIT) to Zeugodacus scutellata, and determining the dose of electron beam irradiation, and a method of controlling Zeugodacus scutellata by releasing the produced sterile males.

### Technical Solution

To achieve the above object,

the present invention provides a method of producing sterile males of Zeugodacus scutellata by irradiating Zeugodacus scutellata pupae with an electron beam at a dose of 150 Gy to less than 250 Gy. More preferably, the Zeugodacus scutellata pupae may be irradiated with an electron beam at a dose of 200 Gy. In addition, the Zeugodacus scutellata pupae are preferably 3- to 5-day-old pupae.

The present invention provides a method for controlling Zeugodacus scutellata comprising steps of: producing sterile males of Zeugodacus scutellata by irradiating Zeugodacus scutellata pupae with an electron beam at a dose of 150 Gy to less than 250 Gy; and releasing the sterile males. Preferably, the sterile males and normal males may be mixed together and released, and more preferably, may be mixed together at a ratio of 9:1 and released. Preferably, the Zeugodacus scutellata pupae may be irradiated with an electron beam at a dose of 200 Gy. The Zeugodacus scutellata pupae are preferably 3- to 5-day-old pupae. In addition, it is more preferable that the sterile males be released again within 2 months after release.

### Advantageous Effects

According to the present invention, it is possible to produce sterile males of Zeugodacus scutellata by using a relatively safe electron beam instead of high-risk radiation used in the conventional sterile insect release technique and irradiating the electron beam at an appropriate dose.

The sterile males of Zeugodacus scutellata produced according to the present invention can maintain the survival rate of adults without much difference from an untreated control for about 3 months, and the laid eggs hardly hatch while the mating rate and the egg laying rate are not significantly lowered. Thus, the sterile males may exhibit a high control effect.

According to the present invention, it is possible to effectively control Zeugodacus scutellata by releasing the produced sterile males of Zeugodacus scutellata together with normal males. At this time, the ratio of the sterile males of Zeugodacus scutellata to normal males is preferably 9:1.

The present invention may also be applied to fruit flies subject to quarantine that will enter Korea in the future.

### BRIEF DESCRIPTION OF THE DRAWINGS

FIG. 1 shows the results of microscopic observation of the ovarian and testis developmental state of Zeugodacus scutellata. In the figure, the scale bar indicates 10 μm.

FIG. 2 shows the results of observing the development of ovaries and testes at certain time points (0 days, 5 days, days, 15 days, 20 days, 25 days and 30 days) after emergence (“DAE”) into Zeugodacus scutellata adults. In the figure, the scale bar indicates 10 μm.

FIG. 3 shows the results of evaluating the effect of electron beam irradiation on development into Zeugodacus scutellata adults. Different letters above the standard deviation bar indicate that there is a significant difference between the means at Type I error=0.05 (LSD test).

FIG. 4 shows the results of evaluating the effect of electron beam intensity on the lifespan of developed Zeugodacus scutellata adults when Zeugodacus scutellata pupae are irradiated with an electron beam.

FIG. 5 shows the results of comparing the mating rate of Zeugodacus scutellata males and untreated females depending on electron beam intensity. The asterisk indicates that there is a significant difference between the means compared at Type I error=0.05 (LSD test). ‘NS’ indicates no significant difference.

FIG. 6 shows the results of evaluating the effect of electron beam irradiation on the egg laying ability of Zeugodacus scutellata adults. ‘NS’ indicates that there is no significant difference between the means compared at Type I error=0.05 (LSD test).

FIG. 7 shows the results of evaluating the effect of electron beam irradiation on the egg hatching rate of the next generation of Zeugodacus scutellata. ‘NS’ indicates that there is no significant difference between the means compared at Type I error=0.05 (LSD test).

FIG. 8 shows the results of examining the death rate after release of electron beam-irradiated Zeugodacus scutellata adults.

FIG. 9 shows the results of examining the number of laid eggs and the hatching rate after release of electron beam-irradiated Zeugodacus scutellata adults.

### DETAILED DESCRIPTION

Prior to the development of sterile insect release technique (SIT), the in vivo reproductive physiological process of Zeugodacus scutellata was analyzed.

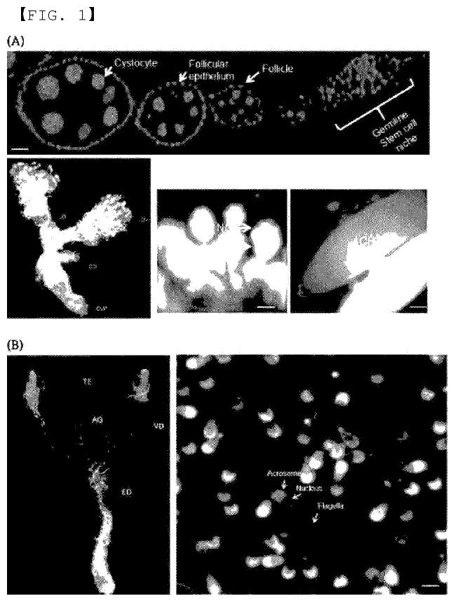

As shown in FIG. 1, in the case of females, a pair of ovaries has about 50 ovarioles, germline stem cells are present at the distal end of each ovariole, and follicles continuously grow therefrom. Each follicle is differentiated into the follicular epithelium surrounding the follicle and nurse cells and oocytes therein. That is, Zeugodacus scutellata has typical polytrophic ovarioles. Regarding the differentiation of oocytes, previtellogenesis (differentiation into stem cells), vitellogenesis (enlargement of oocytes), and choriogenesis (formation of chorion surrounding the oocytes) are all observed with aging of female adults. It can be seen that, about 20 days after emergence, oocytes with chorion are formed and are basically ready for egg laying. That is, the pre-egg laying period of Zeugodacus scutellata at 25° C. is estimated to be about 20 days, even though it depends on the environmental temperature.

A pair of testes of Zeugodacus scutellata has a structure connected to the ejaculatory duct following the common vas deferens. This appearance is also observed in males immediately after emergence. Also, when the part corresponding to the vas efferens is cut at this time and the internal materials are collected, it is possible to observe the appearance of mature sperm. Guillén et al. (2016) presented the morphological criteria for fertile males in Ceratitis capitata by the appearance of fully developed testes and the occurrence of spermatogenesis. Judging from these criteria, it is physiologically presumed that male Zeugodacus scutellata is ready to mate immediately after emergence.

Sterile males of Zeugodacus scutellata are produced by irradiating Zeugodacus scutellata pupae with an electron beam.

As the Zeugodacus scutellata pupae, 3-5-day-old pupae are preferably used. The Zeugodacus scutellata irradiated with the electron beam emerge into sterile males of Zeugodacus scutellata.

An electron beam is used to produce sterile males of Zeugodacus scutellata. An electron beam having energy of 5 to 10 MeV is preferred, and this electron beam has an effect of killing bacteria by cutting the double helix structure of DNA in cells and indirectly forming radicals by ionization of water, etc. (Kwon et al., 2014). It is preferable to irradiate the electron beam at a dose of 200 Gy.

As a result of irradiating electron beams at various doses, a dose higher than 250 Gy caused serious damage to pupa development, thus lowering the emergence rate. At a dose of less than 250 Gy, the mating rate with untreated females and the lifespan of adults decreased as the dose increased, and at 250 Gy, the mating rate was very low and the lifespan was short. Upon irradiation with an electron beam at a dose of 200 Gy, the mating rate was lowered, but the electron beam did not affect the number of eggs laid by the females mating for a certain period of time after emergence, suggesting that normal mating was achieved. It was found that the Zeugodacus scutellata adults irradiated with the electron beam maintained the survival rate of adults for about 3 months, without a significant difference from the untreated control group. In addition, eggs laid by the normal females mated with the males treated with 200 Gy of electron beam did not hatch at all.

A study indicating that the changes in lifespan and mating behavior by irradiation caused changes in the molecular structure related to lifespan and mating behavior was shown through proteomic analysis in Bactrocera orientalis treated with radiation. In this study, it was found that, in the case of treated males, the expression of 35 proteins was disrupted, and these proteins were mainly proteins related to energy metabolism and pheromone sensing (Chang et al., 2015).

Based on these results, in the present invention, Zeugodacus scutellata pupae are irradiated with an electron beam at a dose of 150 Gy to less than 250 Gy to produce sterile males of Zeugodacus scutellata. More preferably, the Zeugodacus scutellata pupae may be irradiated with an electron beam at a dose of 200 Gy. The Zeugodacus scutellata pupae are preferably 3- to 5-day-old pupae.

In addition, in the present invention, Zeugodacus scutellata may be effectively controlled by releasing the sterile males of Zeugodacus scutellata treated with an electron beam at a dose of 150 Gy to less than 250 Gy. More preferably, the Zeugodacus scutellata pupae may be irradiated with an electron beam at a dose of 200 Gy. The Zeugodacus scutellata pupae are preferably 3- to 5-day-old pupae.

Preferably, the sterile males of Zeugodacus scutellata according to the present invention may be released together with normal males. More preferably, the sterile males and normal males may be mixed together at a ratio of 9:1 and released. Since it has been confirmed that the sterile males of Zeugodacus scutellata have a control effect for about 2 months due to their short lifespan, the sterile males are preferably released again within 2 months after release.

Hereinafter, the present invention will be described in more detail with reference to experimental examples. These examples are for illustrating the present invention, and the scope of the present invention is not limited thereto.

### EXPERIMENTAL EXAMPLES

Breeding of Test Insects

The larvae of Zeugodacus scutellata were collected from the flowers of Trichosanthes kirilowii var. japonica native to the Jeju area. The collected larvae were fed pumpkin flowers and proliferated under indoor conditions (temperature: 24 to 27° C.; 16-hr light/8-hr dark cycle; relative humidity: 60 to 80%). During the larval period, pupation was induced by adding bed soil around the food, and to the emerged adults, artificial feed (yeast extract:whole milk powder:sugar:water=2:2:4:1, g/g) and water were supplied.

Statistical Analysis

The bioassay results for emergence rate expressed as percentage were arsine transformed and then analyzed by ANOVA using SAS PROC GLM (SAS Institute, 1989), and comparison between treatment means was performed. On the other hand, for frequency data such as mating rate and hatching rate, a test of independence was performed using PROC FREQ.

### Experimental Example 1

Observation of Reproductive Organs of Zeugodacus Scutellata Adults

The oocyte and sperm structures of Zeugodacus scutellata adults were observed under a fluorescence microscope as follows.

For observation of cell structures using fluorescent substances, fluorescein isothiocyanate (FITC)-tagged phalloidin (Sigma-Aldrich Korea; Seoul, Korea) and DAPI (4′,6-diamidino-2-phenylindole) (Thermo Fisher Scientific; Rockford, Ill., USA) were used.

In order to analyze the growth of ovaries or testes of adults, unmated males and females were arbitrarily selected for each developmental period (0 to 30 days) after emergence under the above breeding conditions.

Ovaries and testes were extracted respectively from female and male Zeugodacus scutellata using 100 mM phosphate-buffered saline (PBS) (pH 7.4) under a dissecting microscope (Stemi SV11, Zeiss, Germany).

For fluorescence analysis, the extracted ovarian and testis tissues were fixed in 3.7% paraformaldehyde for 60 minutes at room temperature under dark conditions, respectively. The fixed tissues were washed three times with PBS, and then reacted with Triton X-100 (dissolved in PBS at a concentration of 0.2%) for 20 minutes. After reaction, the cells were washed three times with PBS and then reacted with 5% skim milk (MB cell, Seoul, Korea) at room temperature for 60 minutes. After reaction, the cells were washed again with PBS and then reacted with FITC-tagged phalloidin at room temperature for 1 hour. After reaction, the cells were washed again with PBS three times, and then the nucleus and cytoplasm were stained with DAPI (1 mg/ml, blue) and phalloidin (green), respectively, for 2 minutes at room temperature. After staining, the stained cells were washed 3 times with PBS and then observed under a fluorescence microscope (DM2500, Leica, Wetzlar, Germany) at 200× magnification.

The ovarian and testis developmental states of Zeugodacus scutellata observed under the microscope are shown in FIG. 1. In FIG. 1, blue showing fluorescence indicates staining of the nucleus with DAPI, and green indicates staining of F-actin with FITC.

The results of observing the female reproductive system are shown in FIG. 1(A). The overall female internal organs include the ovaries (‘OV’), the lateral oviduct (‘LO’), the common oviduct (‘CO’) and the ovipositor (‘OVP’).

Each ovary consists of about 50 ovarioles, germline stem cells exist at the distal end of each ovariole, and follicles are differentiated therefrom. The distal region of the ovariole was stained with DAPI to show follicular development. The initial follicle is surrounded by follicular epithelium and shows the division of cystoblasts to cystocytes. As ovarian development progresses, cystoblasts differentiate into oocytes (‘OC’) and nurse cells (‘NC’). The mature oocyte is then surrounded by the chorion. The FITC-stained region shows nurse cells (‘NC’), oocytes (‘OC’) and chorionated oocytes (‘CH’).

The results of observing the male reproductive system are shown in FIG. 1(B). The overall male internal organs include testes (‘TE’), vas deferens (‘VD’), accessory gland (‘AG’) and ejaculatory duct (‘ED’).

Testes exist as a pair, and each long common vas deferens is gathered in the ejaculatory duct. The accessory gland is then connected to the ejaculatory duct. The testis developed through the vas efferens region existing at the base of the testis was observed under a fluorescence microscope. Each sperm had a head and a long tail, and in the head region, an acrosome was observed in the nuclear region where DNA was present and in the distal region.

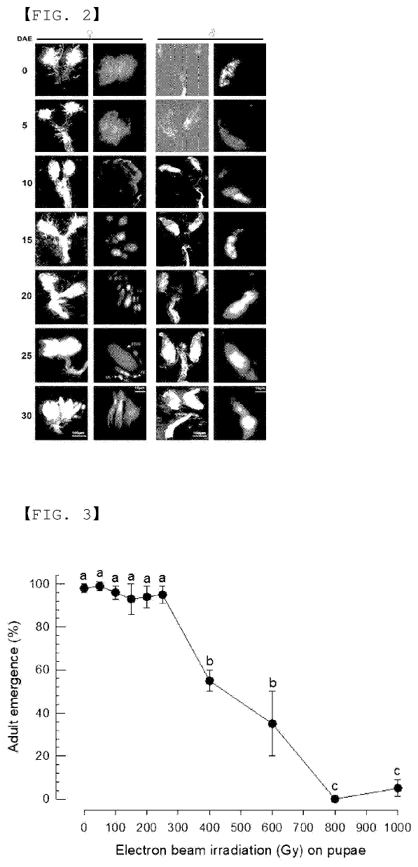

The developmental processes of the ovaries and testes were observed for certain time points (0 days, 5 days, 10 days, 15 days, 20 days, 25 days, and 30 days) after emergence (‘DAE’) into adults, and the results are shown in FIG. 2. Overall development was observed at 50× magnification. F-actin-stained FITC was observed under a fluorescence microscope at 200× magnification.

Immediately after emergence, the ovaries were in a form in which the ovarioles did not yet differentiate and a large number of bronchi were gathered in each ovarian region. 10 days after emergence, hypertrophic growth of the ovary was observed, and after 15 days, the development of the ovarioles could be observed. 20 days after emergence, many oocytes were undergoing vitellogenesis, but some oocytes had begun to form eggs with a chorion. 25 days after emergence, most of the ovarioles had fully developed egg structures so that they could spawn at any time, and the structure of the oocytes showed nurse cells (‘NC’), oocytes (‘OC’) and follicular epithelium (‘FE’).

It was observed that the testis had a complete testis structure immediately after emergence and had a number of DNA-stained structures therein. This appearance was maintained in a similar structure up to 30 days after emergence.

### Experimental Example 2

Analysis of Emergence Rate of Electron Beam-Irradiated Insects

Pupae of Zeugodacus scutellata were irradiated with electron beams at various intensities to form sterile males of Zeugodacus scutellata, and then emergence into adults was observed. The effect of the electron beam on the development of pupae of Zeugodacus scutellata was analyzed as follows.

Pupae of Zeugodacus scutellata after 3 to 5 days of pupation under indoor conditions (25° C.) were irradiated with electron beams at various intensities (0, 100, 200, 300, 400, 500, 600, 700, 800, 900 and 1,000 Gy) and were allowed to develop under the indoor conditions.

Electron beam irradiation was performed using an electron beam device (MB10-8/635, Mevex, Stittsville, Ontario, Canada) in Seoul Radiology Services Co. (Eumseong, Chungbuk). 10 pupae were used for each electron beam treatment, and the treatment was repeated 3 times. After electron beam treatment, the number of emerged adults was counted every day, and the total number of emerged adults for 15 days after treatment was compared and analyzed. The results are shown in FIG. 3.

As can be seen from the results of FIG. 3, when the intensity of electron beam irradiated was 250 Gy or more, the emergence rate significantly decreased as the intensity of the electron beam increased (F=28.29; df=8, 31; P<0.0001). In particular, the electron beam at an intensity of 400 Gy or higher had a serious effect on the development of the pupae and suppressed the emergence of the pupae into adults. However, it was shown that, when the intensity of the electron beam was 250 Gy or lower, there was no difference in development into adults between the treated and untreated groups. The 50% lethal electron beam intensity was estimated to be about 503 Gy.

### Experimental Example 3

Examination of Adult Lifespan of Electron Beam-Irradiated Insects

The effect of the intensity of an electron beam on the lifespan of developed adults when pupae were irradiated with the electron beam was examined as follows.

3- to 5-day-old pupae of Zeugodacus scutellata were irradiated with electron beams at various intensities (0, 200, 400 and 600 Gy), and then the obtained males were placed in containers (11.5 cm diameter×8 cm height), and adult food and water were supplied thereto while being replaced every 3 to 4 days. In the experiment, the following males were used: 21 males irradiated with 0 Gy; 12 males irradiated with 200 Gy; 16 males irradiated with 400 Gy; and 10 males irradiated with 600 Gy.

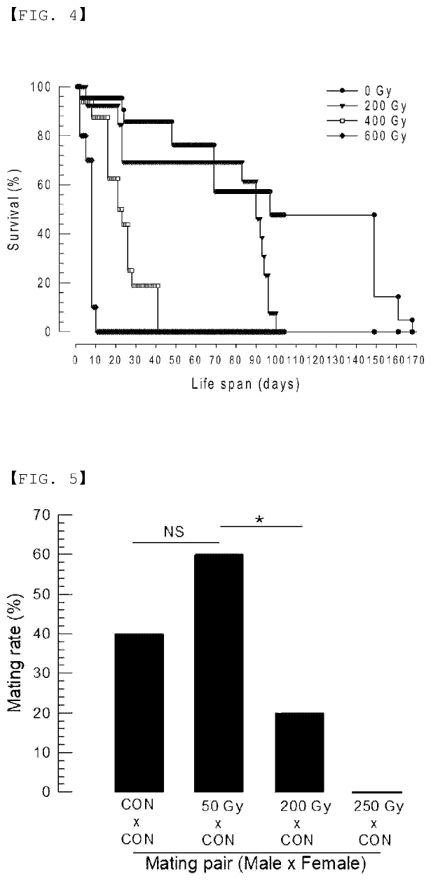

Breeding conditions were as follows: a temperature of 24 to 28° C.; a humidity of 60 to 80%; and a 15-hr light/9-hr dark cycle. Each treatment was repeated with 10 to 21 males. When there was no voluntary activity, the corresponding male was judged to be dead. The results are shown in FIG. 4.

As can be seen from the results in FIG. 4, the lifespan of untreated males was at most about 5 months or longer, but when the males were irradiated with the electron beams, the lifespan of the emerged male adults decreased. This shortening of lifespan became evident as the electron beam intensity increased, and the males treated with the electron beam at 200 Gy mostly showed a survival rate similar to that of untreated males up to 3 months.

### Experimental Example 4

Analysis of Adult Mating Rate, Egg Laying Ability and Hatching Rate for Electron Beam-Irradiated Insects

The mating rate of males depending on the electron beam intensity was examined as follows.

3- to 5-day-old pupae of Zeugodacus scutellata were irradiated with electron beams at various intensities (0, 50, 200 and 250 Gy), and then each of the obtained males was paired with an untreated female (‘CON’) and placed in a container (11.5 cm diameter×8 cm height), and adult food and water were supplied thereto. The food and water were replaced every 3 to 4 days, and pumpkin flower stalks or pumpkin flowers were supplied so that the females could lay eggs. Mating and egg laying were checked daily. Breeding conditions were as follows: a temperature of 24 to 28° C.; a humidity of 60 to 80%; and a 15-hr light/9-hr dark cycle. Each treatment was repeated 5 times. Whether or not egg laying occurred was visually checked by disassembling the flower stalk. Eggs were transferred into Petri dishes (9 cm diameter×3 cm height) and whether or not hatching occurred was checked.

1. Mating Rate

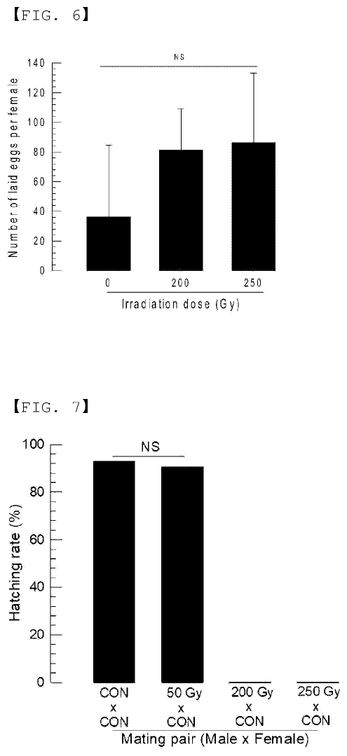

The results of comparing the rate of mating between males and untreated females depending on the electron beam intensity are shown in FIG. 5. The mating rate of untreated males was about 40%, the mating rate of males treated with 50 Gy increased to about 60%, and the mating rate of males treated with 200 Gy decreased to about 20%. On the other hand, the mating rate of males treated with 250 Gy significantly decreased.

2. Egg Laying Ability

In order to evaluate the effect of electron beam irradiation on the egg laying ability of adults, the egg laying ability of females mated with electron beam-irradiated males was compared with that of females mated with untreated males, and the results are shown in FIG. 6. The egg laying ability was determined by the number of eggs laid.

A female mated with an untreated male laid about 40 eggs. However, a female mated with a male treated with a dose of 200 Gy laid about 80 eggs, and a female mated with a male treated with a dose of 250 Gy also showed an egg laying ability similar thereto.

3. Hatching Rate

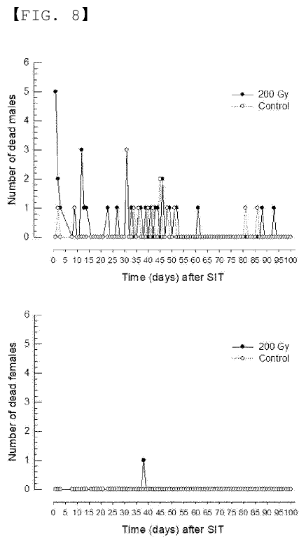

FIG. 7 shows the results of evaluating the effect of electron beam irradiation on the egg hatching rate of the next generation.

The hatching rate of eggs laid by females mated with the electron beam-irradiated males was compared with that of the untreated group. The females mated with untreated males showed a hatching rate of about 90%. Eggs obtained by mating with the males treated with a low dose of 50 Gy also showed a similar hatching rate. However, all eggs obtained by mating with the males treated with a dose of 200 Gy or more did not hatch.

### Experimental Example 5

Analysis of Male Release Technique and Next-Generation Forming Ability of Electron Beam-Irradiated Insects

For a treated group, 36 sterile males obtained by irradiating Zeugodacus scutellata pupae with an electron beam at 200 Gy and 4 untreated males were placed in a box having a certain size (40 cm×40 cm×40 cm), and 4 untreated females were released therein.

For an untreated group (control group), 40 untreated males and 4 untreated females were released into a box having the same size.

1. Death Rate

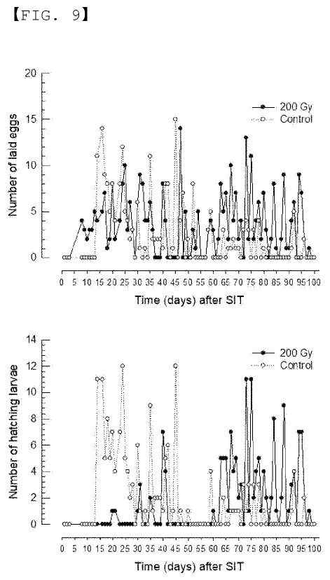

The number of deaths of adults was checked every day after release, and the results are shown in FIG. 8.

As a result of counting adult deaths for about 3 months after release, the death rate of males was higher in the treated group than in the untreated group. However, the death rate of females was almost similar between the untreated group box and the treated group box, and only one female in the treatment group died.

2. Number of Laid Eggs and Hatching Rate

The number of laid eggs and hatching rate were examined in each box for about 10 days after release, and the results are shown in FIG. 9.

As a result of examining the number of laid eggs and hatching rate in the boxes of the treated and untreated groups for about 100 days, it was confirmed that 218 eggs were laid in the untreated group and 54.5 eggs were laid per female. On the other hand, 338 eggs were laid in the treated group and about 84.5 eggs were laid per female.

The overall hatching rate was 76.6% in the untreated group and 45.9% in the treated group. However, as a result of analyzing this difference at each time point, the hatching rate of eggs laid during the initial 2 months after release was 12.7% in the treated group (the sterile males were released) and 77.6% in the untreated group. Since the number of the sterile males was 9 times that of the normal males, the hatching rate was expected to be about 10%, and the result was similar (12.7%) to the expected value.

However, 2 months after release, the hatching rate increased in the treated group and did not significantly differ between the untreated group and the non-treated group. This is believed to be because the lifespan of the sterile males was short, and thus the mating ability of the sterile males was significantly lowered in the late stage. Therefore, it is considered that a more effective control effect can be obtained only when the sterile males are released again within 2 months after release.

1-9: (canceled)

10. A method for producing sterile males of Zeugodacus scutellata comprising a step of sterilizing males of Zeugodacus scutellata without lowering emergence rate by irradiating 3- to 5-day-old pupae of Zeugodacus scutellata with a 5 to 10 MeV electron beam at a dose of 150 Gy to less than 250 Gy.

11. A method for controlling Zeugodacus scutellata comprising a step of sterilizing males of Zeugodacus scutellata without lowering emergence rate by irradiating 3- to 5-day-old pupae of Zeugodacus scutellata with a 5 to 10 MeV electron beam at a dose of 150 Gy to less than 250 Gy.

12. The method of claim 11, further comprising a step of releasing the sterilized males.

13. The method of claim 12, wherein the releasing is performed by releasing the sterilized males mixed with normal males.

14. The method of claim 13, wherein the sterilized males and the normal males are mixed together at a ratio of 9:1 and released.

15. The method of claim 12, further comprising, after releasing the sterilized males, a step of sterilizing males of Zeugodacus scutellata according to the method of claim 11 and further releasing the sterilized males within 2 months.
